(19) United States
(12) Patent Application Publication
(10) Pub. No.: US 2005/0106212 A1
(43) Pub. Date: May 19, 2005

(54) Metallic structures incorporating bioactive materials and methods for creating the same

(76) Inventors: Michael E. Gertner, Menlo Park, CA (US); Mordachay Schlesinger, Pittsburg, PA (US)

Correspondence Address: PRESTON GATES & ELLIS LLP;ATTN: C. RACHAL WINGER, 925 FOURTH AVE, SUITE 9200, SEATTLE, WA, 98104-1158, US

(21) Appl. No.: 10/497,198

(22) PCT Filed: Nov. 27, 2002
(86) PCT No.: PCT/US02/38275

Related U.S. Application Data
(63) Continuation of application No. 10/196,296, filed on Jul. 15, 2002.
(60) Provisional application No. 60/333,523, filed on Nov. 28, 2001.
(60) Provisional application No. 60/364,083, filed on Mar. 15, 2002.
(60) Provisional application No. 60/323,071, filed on Sep. 19, 2001.
(60) Provisional application No. 60/333,523, filed on Nov. 28, 2001.
(60) Provisional application No. 60/364,083, filed on Mar. 15, 2002.

(51) Int. Cl. A61L 2/00; B05D 1/18; A61F 2/06; A61F 2/04

(52) U.S. Cl. 424/425; 427/2.1; 427/430.1; 427/402; 623/1.42; 623/23.71

(57) ABSTRACT

One embodiment of the invention is directed to a method comprising providing an electrochemical solution comprising metal ions and a bioactive material such as bioactive molecules, and then contacting the electrochemical solution and a substrate. A bioactive composite structure is formed on the substrate using an electrochemical process, where the bioactive composite structure includes a metal matrix and the bioactive material within the metal matrix.

### CROSS-REFERENCES TO RELATED APPLICATIONS

This application claims the benefit of the filing dates of the following U.S. Provisional Patent Applications: 60/333,523, filed Nov. 28, 2001, and 60/364,083 filed Mar. 15, 2002. This application also claims the benefit of the filing date of U.S. patent application Ser. No. 10/196,296, filed on Jul. 15, 2002. All of these U.S. Patent Applications are herein incorporated by reference in their entirety for all purposes.

### BACKGROUND OF THE INVENTION

Medical devices encompass a wide array of therapeutic, prophylactic, or diagnostic tools, typically providing certain mechanical, electrical, electromechanical, or other structural properties designed to conduct particular medical procedures on or in a patient's body. Often, as implants in particular (either temporary or permanent, though in particular permanent), medical device designs are also intended to include characteristics that are sufficiently biocompatible to be acceptable by the host body, else the body may reject or otherwise respond to the device with an undesired result. In particular, medical devices often are designed to have surface characteristics such that the device-tissue interactions at these surfaces are optimized. Accordingly, significant research and development into surface modifications and materials to provide optimal results. In particular, coatings have been the topic of significant interest for providing an external surface layer on medical devices in order to achieve the desired device-tissue interface.

Many different medical devices, and related systems and methods, have also been disclosed for locally delivering bioactive materials into or onto various regions of the body, such as lumens, cavities, tissues, or other spaces, structures, or regions. Such bioactive materials include for example drugs (e.g. chemical or biological compounds, etc.) that exhibit therapeutic effects relative to medical conditions, such as short-term therapy drugs as well as long-term therapy, such as hormonal treatment.

Various different types medical device systems and methods have been previously disclosed for locally delivering bioactive materials into remote regions of the body (e.g. lumen, cavity, tissue, or other body region or space) in order to locally achieve the intended therapeutic, prophylactic, or diagnostic effect there.

One particular type of medical device that has become the topic of much research and commercial development for delivering bioactive agents such as drugs is stents. Of particular interest has been endolumenal stents of the types that most typically form cylindrical or tubular walls that are inserted into body lumens and engage their walls to prevent blockage or collapse, e.g. to maintain lumen patency. Such stents are predominantly used in the vascular system, e.g., the coronary, peripheral and cerebrovascular systems. The most common stents in use today are produced from stainless steel or nickel-titanium alloy (e.g. Nitinol™), although different alloys have also been disclosed, such as cobalt-chromium alloys which have been given much attention in recent years. Such endovascular stents are most typically used in percutaneous translumenal interventional procedures to treat diseases such as coronary artery disease, peripheral vascular disease, and cerebrovascular disease.

Stents are used in other body lumens as well, including for example the hepatobiliary system. Indications for hepatobiliary stents include strictures and malignancy. Such stents are often observed to have limited effect as long-term solutions. Permanent metal stents in the hepatobiliary system are placed mostly for palliative treatment and usually in patients who have less than six months to live.

Notwithstanding the various benefits observed with the wide adoption of conventional stenting, various shortcomings have been observed. In one significant regard, unfortunate and harmful medical conditions have been observed in relation to stent implants within lumens, in particular with respect to intravascular stents. One such response is the formation of thrombus on or around a stent, e.g. in the case of intravascular stenting, which may cause local occlusion or release of occlusive thromboembolism causing downstream ischemia. Another significant example is the tendency for a lumen to re-narrow or “restenose” despite stenting. Research into the pathophysiology of “restenosis” in blood vessels has shown that there is smooth muscle cell proliferation and/or thrombosis shortly after a stent is placed within a vessel lumen. At present, the rate of restenosis, or failure, is 20-50% at six months, necessitating re-stenting and/or surgical correction. Over one million procedures are performed per year to open the coronary arteries, even after stents are placed within them.

In recent years, much research and development in the field of stents has been directed toward adapting them to release bioactive materials as anti-restenosis agents in order to prevent the various side effects observed with conventional un-coated stents, such as thrombosis and/or restenosis. These stents are generally referred to as “drug eluting stents.” Several types of anti-restenosis agents have been investigated for use in drug eluting stents, including anti-coagulation agents, though most particularly the type which target smooth muscle cell mitosis, migration, and proliferation as the most significant observed process of restenosis. For example, some stents release drugs such as rapamycin or paclitaxel into surrounding lumenal wall tissues to combat restenosis.

Many different modes have been previously disclosed for adapting stents to release anti-restenosis drugs as drug eluting stents. Certain examples include conventional or specially adapted stents in combination with an outer jacket or other composite of stent plus an additional sleeve or member that holds and releases the drug, such as “covered stents.” Many other recent advances have been directed toward coating the drug onto the outer surface of the stent itself, such as onto the typical networked metal strut scaffolding of the conventional stent designs.

In one particular example, a hydrophobic drug paclitaxel is coated directly onto the outer surface of the stent struts. According to disclosures related to this example, the highly hydrophobic nature of the drug allows the drug to remain on the stent during delivery and implantation at the lesion site without significant “wash-out” in the aqueous blood pool environment. The drug allegedly then passively releases into the wall.

Of significant interest in various drug eluting stents being developed has been coating the outer surface of the networked stent struts with a coating specifically adapted to hold and release anti-restenosis agents of interest. Many such coatings are polymers that perform such function, including degradable polymers that release the drug via degradation of the polymer, or polymers that are adapted to provide diffusion of the drug therefrom into the surrounding liquid environment (e.g. often non-degradable polymers). More specific examples of degradable and non-degradable polymers that have been used in drug eluting stents include without limitation polylactic acid, polyglycolic acid, and polymethylmethacrylate.

Polymer coatings for drug eluting stents have certain limitations, and in some regards problems, associated with drug storage and release medium on stents and on medical devices in general. Various examples of such limitations have been observed.

According to one example, polymeric coatings typically release bioactive materials relatively quickly. While this may be advantageous and desired in many circumstances, for certain intended drug delivery modalities longer time periods for drug elution may be desired than is achievable with such polymer coatings. In another regard, the degradation kinetics of polymers is often unpredictable, in particular from patient to patient. Consequently, it is difficult to predict how quickly a bioactive material in a polymeric medium will be released by such a polymeric medium. If a drug releases from the medium too quickly or too slowly, the intended therapeutic effect may not be achieved.

In another example, many polymeric materials, including the types previously disclosed for stent coating, have been observed to produce an inflammatory response. For example, certain polymeric coatings on stents in vessels have been observed to produce an inflammatory response on the vessel's walls, exacerbating restenosis.

According to another example, adherence of a polymeric material to a substantially different substrate, such as a metallic substrate, e.g. a stent, is difficult to achieve in manufacturing and to maintain in vivo. Mismatched properties such as different thermal and/or mechanical properties between the polymeric material and the underlying substrate (e.g. expansion characteristics of metallic stents) contribute to this difficulty. Inadequate bonding/adhesion between the stent body and an overlying polymeric material may result in the separation of these two stent components over time, an undesirable property in an implanted medical device. In consideration of this limitation, many polymeric coatings must be modified to maximize adherence to the stent, and such modifications often result in compromised ability to hold and release drug.

A further example of the foregoing relates to a two-part polymeric coating previously disclosed for use in drug eluting stents. One part is primarily intended to provide structure to the coating and adherence around stent struts during use; the other part is primarily intended to hold and release the drug. In order to achieve the requisite integrity of the coating on the stent, e.g. during expansion, the first part must have a certain proportion to the second part in the two-part coating. To this end, the volume achievable with the second part of the coating is limited, and thus limiting the amount of drug that can be held and released.

A further limitation of polymer coatings for drug eluting stents is the difficulty to achieve an even coating of a small metallic substrate with a polymeric material. As a small metallic object such as a stent is made smaller (e.g., less than 3 mm in diameter), it becomes more difficult to coat it evenly with a polymeric material. When the polymer is deposited, because it is viscous, it is difficult to evenly coat the object and faithfully replicate its form. This is particularly challenging at various regions of a stent, such as at apices of bends in or bonds between stent struts where viscous materials may accumulate under surface tension. Where unwanted polymer build-up results, folding and expansion characteristics of the coated stent may be compromised. In addition, to the extent the polymer is holding and releasing drug, uneven coating corresponds to uneven and unpredictable drug delivery along and around the stent. To the extent dosing of drug is important to predict along the tissue engaged by the stent, increased or decreased dose from the intended baseline may compromise the intended effects of the drug. Much research and development has been directed toward overcoming this limitation, and in many circumstances significant modifications to or tightly held parameters of the polymeric coating process, or addition of process steps, must be made to achieve acceptably even coatings.

Still a further exemplary limitation of polymeric drug coatings, polymeric storage and release media are typically large and bulky relative to their bioactive material storage capacity. In one regard, stents are designed with particular strut thicknesses and undulating designs so as to maximize mechanical support properties at the vessel wall while minimizing size for profile considerations during delivery to and across a lesion and also to minimize turbulence along the luminal wall in a flowing blood field. Polymer coatings for drug eluting stents are applied over these stent struts, and increase the size of the resulting coated strut. Such increase is directly proportional to the amount of coating necessary to hold the requisite volume of drug for the intended therapeutic or prophylactic effect. It would be desirable if the storage density of bioactive material storage medium could be increased so that an intended volume of bioactive material could be released, often over a long period of time, while minimizing the bulk of the release media.

According to another significant limitation, polymeric coatings are typically limited in their ability to be processed with, hold, or release bioactive agents of only particular types. Whereas bioactive agents may be hydrophobic, hydrophilic, organic, inorganic, or otherwise distinguishable in structure and activity, polymeric coatings often are suitable for the desired interactions with only certain species of these classes. Therefore, certain drugs may not work with a particular coating, and certain combinations or “cocktails” of multiple drugs can not be coated onto the same substrate using the same coating. However, it would be desirable for a coating to work with a wide variety of types of bioactive agents, in particular where a “cocktail” of multiple agents is desired to be coated onto the same substrate such as a stent.

Still further limitations of polymeric coatings for medical devices abound. For example, when delivering a bioactive material to a patient over a longer time period, particularly in an in vivo environment, the bioactive material needs to be stabilized. Some polymeric materials may not provide for a stable storage environment for the bioactive material, in particular when liquid is able to seep into the polymeric material. In another example, polymers having relatively large pores can protect micro-organisms in the interstices of the polymeric release medium, thus increasing the risk of infection. In another example, certain polymeric coatings requiring processing parameters and/or materials (e.g. temperatures, solvents, or other aspects) which may be harmful to the intended drug to be held and released by the coating. Thus, multiple steps must be included in the manufacturing process of the drug eluting stent or other medical device in order to get the polymer coated and the drug into the polymer. According to yet another example, polymer coatings currently under development contribute bulk but do not contribute to the major function of the stent, which is to provide a structural support to prop open the body lumen. It would further be desirable if the storage medium for the bioactive material contributed to the mechanical strength of the object.

Furthermore, underlying substrates to be coated often require electrical surface conductivity, such as in the case of electrodes - many polymers do not provide for such conductivity, and such polymers may not be suitable to hold and release certain drugs. Alternatively, polymer coatings must be modified to provide for such conductivity, which may impact the other intended characteristics and complexity. Still further, coated devices may benefit from enhanced radiopacity wherever possible, such as for example nickel-titanium (e.g. NiTi stents). Most polymer coatings, in particular for drug eluting stents or otherwise coating bioactive agents, do not provide this benefit or otherwise would require significant modification to provide for radiopacity. It would be desirable for a medical device coating, in particular that is adapted to hold and/or release bioactive agents, to provide further benefits such as electrical surface conductivity or radiopacity.

Notwithstanding the significant prevalence of polymeric coatings in drug eluting stent research and development, other modalities have also been disclosed for adapting stents and other medical devices to hold and release bioactive agents. In one regard, certain prior disclosures attempt to deposit drugs into wells, grooves, or other cavities or reservoirs formed into the surface of the medical device itself for holding and releasing bioactive agents such as drugs. Other examples of coatings that have been disclosed for use in drug eluting stents in particular include for example ceramics and hydrogels.

At least one additional disclosed example provides a sintered metallic structure intended to provide a porous surface for delivering a therapeutic agent. Sintering generally involves fusing small particles of metal using heat and/or pressure to weld them together. Porous sintered metallic structures typically have relatively large pores. When a bioactive material is loaded into the pores of a sintered metallic structure, the larger pore size can cause the biologically active material to be released very quickly. Also, because a relatively high temperature is used to form a sintered structure, a bioactive material including biologically active molecules generally must be loaded into the sintered structure after the porous structure is formed, whereas “co-deposition” is often not possible as the bioactive agent would denature or otherwise be damaged from the heat. This method is generally time consuming, and may in some circumstances be difficult to impregnate the pre-formed pores of the sintered structure with certain biologically active molecules. Consequently, it is difficult to fully load the sintered structure with them. When impregnating a sintered structure, the bioactive molecules may be in a carrier such as water or other substance. The surface tension of the carrier may preclude the biologically active molecules from thoroughly impregnating the sintered structure. As a result, the sintered structure may not be fully loaded with the biologically active molecules.

For further illustration, one previously disclosed example is intended to load a therapeutic agent in a fluid form into a previously sintered stent by immersing the sintered stent in a medicated solution. The therapeutic agent may be dissolved in a solvent or suspended in a liquid mixture. An average pore size that is more than ten times the particle size of a suspended therapeutic agent is an intended result of sintering according to this disclosure. Moreover, use of pressure is further disclosed to aid the passage of medicated fluid into the porous cavities of the stent.

As noted above, it would be desirable to have ability to increase the bioactive material storage capacity in a bioactive composite material so that, for example, the bioactive material can be released to a patient over a long period of time. In another regard, because a liquid (blood, water, etc.) can enter into the pores of the material, the stability of the bioactive materials is limited. Such high temperatures also render this process incompatible with certain underlying substrates that may be structurally or functionally degraded by the heat exposure, such as for example certain polymeric and other material substrates, and in particular nickel-titanium substrates (e.g. NiTi stents), which have trained material properties such as superelasticity or shape memory that might be diminished under the heat exposure.

Still further previously disclosed coating examples use electroplating methods for coating metals onto surfaces, such as onto substrates to form or coat medical devices. Electroplating generally involves exposing a surface to an environment that includes metal particles. An electrical charge or current is applied and results in deposition of the metal onto the surface. While electroplating metals to form structures associated with medical devices may provide benefits in certain situations, in certain circumstances it would be beneficial if such metal deposition could be achieved without requiring the formation of an electrical circuit and/or application of electrical current.

Further examples of coatings intended for use for drug eluting stents require formation of multiple coating materials, such as in multiple layers on a substrate. In one such example, one layer may be used for adhesion to a substrate, the other for holding and releasing drug. In another example, one coating may hold one type of bioactive material, the other holds another type. In another example, one coating layer holds drug onto a stent, an additional top layer envelops the first layer and provides for delayed release of the drug not otherwise achievable via the first layer. Another example provides what is intended to be a biomimetic coating with multiple layers intended to be mimic cell wall structures intended to enhance biocompatibility of the coated surface.

These other examples of previously disclosed modes for coating or adapting medical devices for release of bioactive agents suffer from respective various limitations similar to one or more of those provided above with respect to polymeric coatings, including without limitation: processing limitations in relation to underlying substrate or bioactive agent to be coated; even distribution of coating or drug; adhesion; biocompatibility (e.g. toxicity, or other adverse biological response); complexity of processing; size; density and thus volume of drug that can be held and released; timing of drug release; high electrical impedance; low radiopacity; or impact of the coating on the underlying substrate's intended function (e.g. mechanical properties, expansion characteristics, electrical surface conduction, radiopacity, etc.).

Further more detailed examples of medical devices or other structures or methods providing general background with respect to this description are variously disclosed in the following issued U.S. Patents: U.S. Pat. No. 4,358,922 to Feldstein; U.S. Pat. No. 4,374,669 to Mac Gregor; U.S. Pat. No. 4,397,812 to Mallory, Jr.; U.S. Pat. No. 4,547,407 to Spencer, Jr.; U.S. Pat. No. 4,729,871 to Morimoto; U.S. Pat. No. 4,917,895 to Lee et al.; U.S. Pat. No. 5,145,517 to Feldstein et al.; U.S. Pat. No. 5,338,433 to Maybee et al.; U.S. Pat. No. 5,464,524 to Ogiwara et al.; U.S. Pat. No. 5,616,608 to Kinsella et al.; U.S. Pat. No. 5,624,411 to Tuch; U.S. Pat. No. 5,700,286 to Tartaglia et al.; U.S. Pat. No. 5,725,572 to Lam et al.; U.S. Pat. No. 5,772,864 to Moller et al.; U.S. Pat. No. 5,843,172 to Yan et al.; U.S. Pat. No. 5,873,904 to Ragheb et al.; U.S. Pat. No. 5,958,430 to Campbell et al.; U.S. Pat. No. 5,972,027 to Johnson; U.S. Pat. No. 5,976,169 to Imran; U.S. Pat. No. 6,019,784 to Hines; U.S. Pat. No. 6,042,875 to Ding et al.; U.S. Pat. No. 6,123,861 to Santini, Jr. et al; 6,143,037 to Goldstein et al.; U.S. Pat. No. 6,174,329 to Callol et al.; U.S. Pat. No. 6,180,162 to Shigeru et al.; U.S. Pat. No. 6,231,600 to Zhong; U.S. Pat. No. 6,240,616 to Yan; U.S. Pat. No. 6,253,443 to Johnson; U.S. Pat. No. 6,258,121 to Yang et al.; U.S. Pat. No. 6,273,913 to Wright et al.; U.S. Pat. No. 6,280,411 to Lennox; U.S. Pat. No. 6,287,249 to Tam et al.; U.S. Pat. No. 6,287,285 to Michal et al.; U.S. Pat. No. 6,306,166 to Barry et al.; U.S. Pat. No. 6,309,380 to Larson et al.; U.S. Pat. No. 6,315,794 to Richter; U.S. Pat. No. 6,322,847 to Zhong et al.; and U.S. Pat. No. 6,447,664 to Taskovics et al. The disclosures of these references are herein incorporated in their entirety by reference thereto.

Additional examples are also variously disclosed in the following U.S. Patent Application Publications: U.S. 2001/0032014 to Yang et al.; and U.S. 2002/0098278 to Bates et al. The disclosures of these references are herein incorporated in their entirety by reference thereto.

Still further examples are variously disclosed in Published PCT Patent Applications having the following International Publication Numbers: WO 89/03232 to Bar-Shalom et al.; WO 91/12779 to Wolff et al.; WO 91/17286 to Tarasevich et al.; WO 93/19803 to Heath et al.; WO 98/36784 to Ragheb et al.; WO 99/08729 to Barry et al.; WO 99/25272 to Richter et al.; WO 00/10622 to Ragheb et al.; WO 00/21584 to Barry et al.; WO 00/27445 to Boock et al; WO 00/29501 to Hampikian et al.; WO 00/32238 to Palasis et al.; WO 00/32255 to Kamath et al.; WO 01/01890 to Yang et al.; WO 01/14617 to Leclerc et al.; WO 01/15751 to Ahola et al.; WO 01/70294 to Eidenschink et al.; WO 01/87372 to Kopia et al.; and WO 02/058775 to Segal et al. The disclosures of these references are herein incorporated in their entirety by reference thereto.

Still further examples are disclosed in the following Published European Patent Applications: 0 568 310 to Mitchell et al.; EP 0 734 721 to Eury et al.; EP 0 747 069 to Fearnot et al.; EP 0 950 386 to Wright et al. The disclosures of these references are herein incorporated in their entirety by reference thereto.

Notwithstanding certain benefits that may be provided by the foregoing examples for forming or coating structures for use in medical devices, it would be beneficial if a coating process and matrix could be provided that overcomes one or more of the limitations of these prior attempts, such as for example (but without limitation) being able to provide smaller and/or more densely packed surface pores in certain circumstances, to deposit a bioactive material in the coating during the coating process, to process at reduced temperatures, to provide predictable and even coating coverage on substrates, to provide improved adhesion on difficult substrates (e.g. nickel titanium), etc.

The present invention addresses the various limitations and needs that still exist in view of the previous attempts noted above and otherwise, individually and collectively.

### SUMMARY OF THE INVENTION

Certain aspects of the invention are directed to structures, methods, and devices that include a metallic matrix including a bioactive material (e.g., a drug). In some modes according to these aspects, the bioactive material is contained within a metallic matrix. In some embodiments, the matrix can be crystalline and can have grain boundaries. Diffusion of the bioactive material according to these embodiments can occur for example along the grain boundaries and crystallites of the metal. The bioactive material can be within, for example, nanometer and sub-nanometer sized regions within the metallic matrix, such as in void regions. In certain embodiments, the bioactive material can be stored in a metallic matrix and can then be released from the metallic matrix. The bioactive material may diffuse through the metallic matrix or the metallic matrix could erode (actively and/or passively) to release the bioactive material over time. This can be done without using a polymeric storage and release medium for the bioactive material.

One embodiment according to these aspects is directed to a method comprising: (a) providing an electrochemical solution comprising metal ions and bioactive materials; (b) contacting the electrochemical solution and a substrate; and (c) forming a bioactive composite structure on the substrate using an electrochemical process, wherein the bioactive composite structure includes a metal matrix and the bioactive molecules within the metal matrix.

Another embodiment according to these aspects is directed to a bioactive composite structure comprising: (a) a metal matrix, wherein the metal matrix is formed using an electrochemical process; and (b) bioactive molecules within the metal matrix.

Another embodiment according to these aspects is directed to a medical device comprising: a bioactive composite structure comprising a first material, a second material derived from a reducing agent relative to the first material, and a bioactive material. The second material, may be, for example, phosphorous that is derived from a reducing agent such as sodium hypophosphite. In some embodiments, the first material is a metallic material (e.g., nickel, cobalt, etc.) and the first metallic material and second material may form a metallic matrix which incorporates the bioactive material.

Other aspects of the invention are directed to various medical devices that incorporate the bioactive composite structure or are wholly comprised of the bioactive composite structure.

Other aspects of the invention are directed to methods of using the bioactive composite structure.

Another aspect of the invention provides a medical device having a substrate and a coating on the substrate that comprises nickel. According to one mode of this aspect, the substrate also comprises nickel. According to one highly beneficial embodiment of this mode, the substrate comprises a nickel-titanium alloy. According to another embodiment, the coated substrate is characterized as releasing substantially less nickel in an aqueous environment than is released by the nickel-containing substrate alone without the nickel-containing coating. According to one highly beneficial variation of this embodiment, the coated substrate is characterized as releasing at least twenty-five percent less nickel than the uncoated substrate. In another variation, the coated substrate is characterized as releasing at least fifty percent less nickel than the uncoated substrate. According to another mode of this aspect, the substrate comprises a stent. According to one embodiment of this mode, the stent comprises a network of interconnected nickel-titanium struts. According to another embodiment of this mode, the stent comprises a network of interconnected struts constructed from a nickel-titanium alloy

Another aspect of the invention provides an endolumenal stent having a stent wall with an outer surface and a coating on the stent wall that comprises a metal, a reducing agent of the metal, and a bioactive agent. According to one mode of this aspect, the metal comprises a bi-valent metal ion in aqueous solution. According to another mode, the metal comprises a tri-valent metal ion in aqueous solution.

Another aspect of the invention is a medical device having a substrate with an outer coating that comprises a first material, a second material, and a bioactive agent, wherein the first and second materials are characterized as forming cations and anions sufficient to form an electrochemical deposition process when in an aqueous solution.

Another aspect of the invention is a method for coating a medical device comprising: providing a substrate with an outer surface; and forming a coating layer onto the outer surface of the substrate with a coating material while depositing a bioactive agent within the coating layer. One mode of this aspect further includes: releasing the bioactive agent from the coating layer. One embodiment of this mode further includes: while releasing the bioactive agent from the coating layer, substantially maintaining the coating material in the coating layer. Another mode of this aspect includes forming the coating layer without substantially heating the outer surface which in one embodiment includes not heating the outer surface above 120 degrees Fahrenheit. Another mode of this aspect includes forming the coating layer without using a polymeric material. Another mode of this aspect includes: forming the coating material with a first material and a second material that is a reducing agent of the first material (i.e. transfers electrons to the first material). One embodiment of this mode includes providing a metal ion as the first material, and providing a negative ion as the second material which can transfer negative charge to the first material in order to reduce it to the non-charged state.

Another mode of this aspect includes forming a solution of a first coating material, a second coating material, and the bioactive material, wherein the first and second coating materials together form the coating material in the coating layer. One embodiment of this mode further includes contacting the solution with the substrate. One variation of this embodiment includes submerging the substrate within a bath of the solution. Another further highly beneficial variation of this embodiment includes passively forming the coating layer with the solution contacting the substrate.

Another aspect includes a solution that is useful in coating a substrate such as a medical device, comprising: a solution of at least one coating material and at least one bioactive material. According to one mode of this aspect, the at least one coating material comprises a metal ion. According to another mode of this aspect, the bioactive material comprises an anti-restenosis agent. According to one beneficial embodiment of this mode, the anti-restenosis agent comprises at least one of: anti-proliferative agent, anti-mitotic agent, anti-migration agent, anti-inflammatory agent, adhesion inhibitor, platelet aggregation inhibitor, or anticoagulant agent. According to another mode, the solution comprises an aqueous liquid, and the at least one coating material comprises a first material that is an anion and a second material that is a cation in the aqueous liquid. According to one further embodiment of this mode, the first and second materials are adapted to form an electrochemically deposited film on the substrate. In one highly beneficial variation of this embodiment, the first and second materials are adapted to form an electrolessly, electrochemically deposited film on the substrate.

Another aspect of the invention includes a medical device with a substrate and a coating layer on the substrate that increases the radiopacity of the medical device. In one mode, the coating layer includes a metal that increases the radiopacity of the medical device. In another mode, the substrate is a metal substrate. In another mode, the substrate is a stent. In another mode, the coating layer includes a first coating material and a second coating material, wherein at least one of the first and second coating materials increases the radiopacity of the substrate. In another mode, the coating layer includes a bioactive material. In one embodiment of this mode, the coating layer further includes first and second coating materials in combination with the bioactive material. In one highly beneficial further variation of this embodiment, the coating layer is a composite matrix with the first and second coating materials and the bioactive material. In still a further feature of this embodiment, at least one of the first and second coating materials may be a metal. In a further feature that may be beneficially included for this composite matrix variation, the substrate is a stent and the bioactive material is an anti-restenosis material.

Another aspect of the invention is a medical device with an outer surface that includes a non-sintered composite metallic matrix that includes at least one metal and a bioactive material. The medical device matrix is adapted to release the bioactive material within the body.

Another aspect of the invention is a medical device with an outer surface that includes a metal matrix and pores containing bioactive material that are less than about 1 micron in diameter. In one mode, the bioactive material is an anti-restenosis material. In another mode, the medical device comprises a stent and the outer surface is located on the stent struts. In another mode, the pores are less than about 100 angstroms in diameter.

Another aspect of the invention is a medical device with a substrate that includes a metal and a coating on the substrate that includes the same metal. In one mode of this aspect, the metal is nickel. In one embodiment of this mode, the substrate is a nickel-titanium alloy. In one further variation of this embodiment, the coating does not contain titanium. In another mode, the metal is cobalt. In one embodiment of this mode, the substrate contains cobalt and chromium. In one variation that may be beneficially applied to this embodiment, the coating contains both cobalt and chromium. In another mode, the substrate includes an alloy of the metal and a second metal, and the coating does not include the second metal. In another mode, the coating includes a bioactive agent. In one highly beneficial embodiment of this mode, the bioactive agent is an anti-restenosis agent. In another highly beneficial mode, the substrate is a stent.

Another aspect of the invention is a medical device with a substrate and a coating on the substrate that is adapted to contain a variety of types of bioactive materials. In one mode, the coating is adapted to contain either or both of water soluble or water insoluble bioactive materials. In another mode, the coating is adapted to contain either or both of organic or inorganic materials. In another mode, the substrate is a stent. In another mode, at least one type of the variety of bioactive materials is contained within the coating.

Another aspect of the invention is a medical device with a substrate and a coating on the substrate that includes a metal matrix. The metal matrix includes a metal and also is also characterized according to at least one of the following characteristics: a bioactive material is in the metal matrix; or (ii) a relatively radiopaque material relative to the substrate is in the metal matrix; or (iii) the metal matrix is a non-sintered, non-electroplated, non-radioactive metal matrix; or (iv) the metal matrix is an electroless electrochemically deposited metal matrix; or (v) a material derived from a reducing agent of a metal ion formed by the metal in an aqueous fluid is in the metal matrix.

Such a medical device according to this aspect that has a coated substrate exhibiting any one of these characteristics is considered a highly beneficial independent aspect of the invention, whereas combinations incorporating all or any two or more of these characteristics are further considered independently beneficial aspects. Accordingly, one beneficial aspect of the invention is a medical device with a substrate that is coated by a metal matrix having a bioactive material is in the metal matrix. Another beneficial aspect is a medical device with a substrate that is coated by a metal matrix having a relatively radiopaque material relative to the substrate is in the metal matrix. Another beneficial aspect is a medical device with a substrate that is coated by a metal matrix that is non-sintered, non-electroplated, non-radioactive. Another beneficial aspect has a metal matrix coating that is electroless electrochemically deposited, and another aspect is a metal matrix coating that includes a first metal material and a second material that is derived from a reducing agent of a metal ion formed by the metal in an aqueous fluid solution.

Another aspect of the invention is a medical device with a substrate formed from at least two metals and a coating on the substrate. The coating is further characterized as having at least one of the following characteristics: (i) the coating includes a first one of the two metals in the substrate, and exhibits a substantially reduced rate of release of this first metal than would be released from the substrate alone in a blood environment; or (ii) the coating includes a first one of the two metals found in substrate, but does not include the second one of the two metals.

Such a medical device according to this aspect that has a coated substrate exhibiting any one of these characteristics is considered a highly beneficial independent aspect of the invention, whereas combinations incorporating all or any two or more of these characteristics are further considered independently beneficial aspects of the invention. Therefore, a beneficial aspect of the invention is a medical device with a substrate and a coating on the substrate that includes a first one of two metals in the substrate, and exhibits a substantially reduced rate of release of this first metal than would be released from the substrate alone in a blood environment. Another beneficial aspect is a medical device with a substrate that is coated by a coating that has a first one of two metals found in substrate, but which coating does not include the second one of the two metals

In one mode according to this aspect, the two metals in the substrate comprise the two most prevalent materials in the substrate. In another mode, the two metals comprise two principal metals in a metal alloy that makes up the substrate. In further modes, other metals may be further provided in the substrate or coating.

Another aspect of the invention is a medical device that includes a substrate and a bioactive material. The substrate has an outer surface that is at least in part metal, and also has a plurality of regions that are adapted to contain the bioactive material and to release the bioactive material from the substrate in the body of a patient. The bioactive material is contained within the regions. The medical device according to this aspect is further characterized according to at least one of the following characteristics of the regions in the outer surface: (i) they are sufficiently small to substantially prevent water penetration into the bioactive material contained therein when the outer surface is exposed to a blood environment in a patient; or (ii) they have a diameter of less than about 1 micron in diameter; or (iii) they have a diameter that is less than about ten times the size of the bioactive material. Such a medical device that has a coated substrate exhibiting any one of these characteristics is considered a highly beneficial independent aspect of the invention, whereas combinations incorporating all or any two or more of these characteristics are further considered independently beneficial aspects.

Another aspect of the invention is a medical device that includes a bioactive composite structure with a metal matrix and a bioactive material in the metal matrix. The bioactive composite structure forms at least a portion of a stent.

Another aspect of the invention is a medical device that includes a substrate and a coating on the outer surface of the substrate. The coating according to this aspect is characterized as having one or more of the following characteristics: (i) the coating has a thickness over the outer surface of the substrate that is less than about 5 microns and a therapeutic level of bioactive material in the coating; or (ii) the coating includes a metal matrix and a bioactive material in the metal matrix; or (iii) the coating includes a non-electroplated metal matrix. The coated substrate according to this aspect is further characterized as forming at least a portion of a stent.

Such a medical device according to this aspect that has a coated substrate exhibiting any one of the characteristics just described is considered a highly beneficial independent aspect of the invention, whereas combinations incorporating all or any two or more of these characteristics are further considered independently beneficial aspects.

Another aspect of the invention is a method for forming a medical device at least in part by forming a metal matrix according to a process that includes one or more of the following: (i) electroless electrochemical deposition of a metal and a second material derived from a reducing agent with respect to metal ions formed by the metal when in an aqueous solution; or (ii) forming the metal matrix while depositing a bioactive agent in the metal matrix; or (iii) forming the metal matrix as a coating on a substrate without using an applied electrical current and without sintering, or (iv) forming the metal matrix as a coating on a substrate without using an applied electrical current and at a temperature that is less than about 120 degrees Fahrenheit. The method according to this aspect further includes forming the metal matrix as at least a portion of the medical device.

Such a method according to this aspect that includes a process for forming a metal matrix that exhibits any one of the characteristics just described is considered a highly beneficial independent aspect of the invention, whereas combinations incorporating all or any two or more of these characteristics are further considered independently beneficial aspects.

Another aspect of the invention is a method for manufacturing a medical stent at least in part by forming a metal matrix with a process that includes one or more of the following: (i) forming the metal matrix as a coating on a substrate without using an applied electrical current, or (ii) depositing a bioactive material in the metal matrix. The method according to this aspect further includes forming the metal matrix as at least a portion of the stent. Further modes of this method include performing the process without sintering, or in a temperature environment that is less than about 120 degrees Fahrenheit.

Another aspect of the invention is a solution for use in forming at least a portion of a medical device. The solution according to this aspect includes a bioactive material in combination with another material within a fluid that is adapted to form an electrochemical deposition of the bioactive material and the other material onto a substrate contacted by the solution.

Various further modes of the invention that are beneficial further alternative embodiments of the aspects provided above include in one beneficial example forming metal matrix structures in medical devices that include at least one of nickel, cobalt, or chromium in combination with at least one of phosphorous or boron. In more particular beneficial embodiments, nickel is provided in combination with phosphorous in a metal matrix (e.g. as an outer surface of a medical device such as a stent), or cobalt and chromium may be provided in a metal matrix with phosphorous or boron.

Other various modes that may be also incorporated with the aspects above as further embodiments include combination of electroless electrochemical deposition with other deposition methods and/or resulting structures, such as for example sintering and/or electroplating metals in combination with electroless electrochemical deposition of metal matrices. For example, multiple layers of metal matrices may be formed by these various processes with a result providing beneficial composite structures. In still further examples, polymers, ceramics, hydrogels, or other coating materials and related processes may be combined with the various aspects above as further embodiments and variations that provide further independent benefit according to the invention.

Highly beneficial further modes applicable to the various aspects above provide a medical device in the form of an implantable stent. In exemplary embodiments, the stent includes the substrate in the form of struts that are interconnected in a network that forms an expandable tubular body adapted to hold a lumen open in the expanded condition. Various of the coatings, metal matrices, and substrates embodied by the various independent aspects have particularly beneficial application according to such stent modes.

Although medical devices such as stents are discussed in detail, it is understood that embodiments of the invention are not limited to stents or for that matter, to macroscopic devices. For example, embodiments of the invention could be used in any device or material, regardless of size and includes artificial hearts, plates, screws, “MEMS” (microelectromechanical systems), and nanoparticle based materials and systems, etc. Other examples of medical devices and materials according to embodiments of the invention are described below.

These and other aspects, modes, embodiments, variations, and features of the invention are described in further detail with reference to the Figures and the Detailed Description.

### BRIEF DESCRIPTION OF THE DRAWINGS

FIG. 1 shows a schematic illustration of a substrate and a bioactive composite structure on the substrate.

FIG. 2 shows a schematic illustration of a portion of a bioactive composite structure containing a bioactive material.

FIG. 3 shows a device including a bioactive composite structure in between a substrate and a topcoat.

FIGS. 4(a)-4(c) show a stent being placed into a coronary artery.

FIG. 5 shows a flowchart illustrating an exemplary method according to an embodiment of the invention.

FIG. 6 shows a graph showing drug elution profiles associated with Johnson and Johnson Bx velocity stents (stainless steel) with bioactive composite structures according to embodiments of the invention.

FIG. 7 shows a graph showing drug elution profiles associated with stents made with nickel-titanium alloy and bioactive composite structures according to embodiments of the invention.

### DETAILED DESCRIPTION

### I. Definitions

Some terms that are used herein are described as follows.

The terms “anti-restenosis” as herein used in relation to compounds, agents, or other materials generally refer to those “bioactive materials” (as defined immediately below) that at least in part contribute to prevention or inhibition of a restenosis response to vascular injury related to an endolumenal intervention, such as angioplasty, atherectomy, stenting or other recanalization or endolumenal implant procedure. Examples of anti-restenosis agents include anti-mitotic agents, anti-proliferative agents, anti-migratory agents, anti-inflammatory agents, anti-thrombin agents (e.g. thrombin inhibitors), anti-platelet aggregation agents (e.g. platelet adhesion/aggregation inhibitors), healing agents such as endothelialization promoters, or other agents mitigating, preventing, or otherwise intervening in the biological restenosis process.

The terms “bioactive material(s)” refer to a compound, agent, or any other material that exhibits biologically relevant activity on or within a biological organism, including in particular activity that provides treatment, prophylaxis, or diagnosis of a medical condition related to a body of a patient, such as dysfunctional or abnormal conditions associated with the body's structures or functions, or conditions resulting from or otherwise related to a medical procedure, e.g. a medical intervention.

Examples of bioactive materials include drugs for contraception and hormone replacement therapy, and for the treatment of diseases such as osteoporosis, cancer, epilepsy, Parkinson's disease and pain. Further examples of bioactive materials include, without limitation: anti-inflammatory agents, anti-infective agents (e.g., antibiotics and antiviral agents), analgesics and analgesic combinations, antiasthmatic agents, anticonvulsants, antidepressants, antidiabetic agents, antineoplastics, anticancer agents, antipsychotics, and agents used for cardiovascular diseases, such as anti-restenosis compounds and anticoagulant compounds. Further examples of molecules useful as bioactive materials include: hormones, growth factors, growth factor producing virus, growth factor inhibitors, growth factor receptors, antimetabolites, integrin blockers, or complete or partial functional in-sense or anti-sense genes.

The following are further examples of different types of compounds that may be bioactive materials: inorganic, organic, or organometallic; hydrophilic or lipophilic; hydrophobic or lipophobic; water soluble or water insoluble; peptides or proteins; polypeptides; polysaccharides (e.g. heparin); oligosaccharides; mono- or disaccharides; whereas any of the foregoing labels apply with respect to molecules, compounds, or other preparations or materials. Other examples include: living material, such as living or scenescant cells, bacterium, virus, plasmids, genes, other genetic material, or other components or parts thereof; and man-made particles or other materials, for example carrying a biologically relevant or active material.

Bioactive materials may also include precursor materials that exhibit the relevant biological activity after being metabolized, broken-down (e.g. cleaving molecular components), or otherwise processed and modified within the body. These may include such precursor materials that might otherwise be considered relatively biologically inert or otherwise not effective for a particular result related to the medical condition to be treated prior to such modification.

Combinations, blends, or other preparations of any of the foregoing examples may be made and still be considered bioactive materials within the intended meaning. Aspects of the present invention directed toward bioactive materials may include any or all of the foregoing examples.

The term “electrochemical deposition” refers to both electrodeposition (electroplating) and electroless deposition (see method descriptions below).

The term “medical device” refers to a device or structure that is foreign to a body of a living being, such as in particular a human body, but which is adapted for use in performing a therapeutic, prophylactic, or diagnostic function inside, on, or otherwise in relation to the body of a living being, such as in particular human beings. Medical devices include for example many different types of permanent or temporary implants. Further illustrative examples of medical devices include but are not limited to: catheters; guidewires; coils; expandable member devices (e.g. balloons or cages); drug delivery apparatuses, including for example patches; vascular conduits, e.g. grafts, stent-grafts, fistulas; stents; grafts; plates; screws; spinal cages; dental implants; dental fillings; braces; artificial joints; embolic devices; ventricular assist devices; artificial hearts; heart valves; embolic filters (e.g. venous); staples; clips; sutures; prosthetic meshes; mapping; ablation or stimulating electrode devices; pacemakers; pacemaker leads; defibrillators; neurostimulators; neurostimulator leads; intrauterine devices (“IUD's”); syringes; shunts; cannulas; and implantable or external sensors. Medical devices are not limited by size and include micromechanical systems, and nanomechanical systems which perform a function in or on the surface of the human body. Embodiments of the invention include such medical devices.

The term “implants” refers to a category of medical devices, which are implanted in a patient for some period of time. They can be diagnostic or therapeutic in nature, and long or short term, permanent or temporary.

The term “self-assembly” refers to a nanofabrication process to form a material or coating, which proceeds spontaneously from a set of ingredients. A common self-assembly process includes the self-assembly of an organic monolayer on a substrate. One example of this process is the binding of linear organic molecules to a substrate. Each molecule contains a thiol group (S-H moiety). The thiol group of each molecule couples to the gold surface while the other end of the molecule extends away from the gold surface. The process of electroless deposition, which continues spontaneously and auto-catalytically from a set of ingredients, may also be considered a self-assembly process.

The term “stents” refers to medical devices that are adapted to engage the wall of a body lumen or interstitial tract in order to affect the patency thereof, and may be either permanent or temporary implants. Stents are generally adjustable between a radially collapsed condition (e.g. for endolumenal delivery through a delivery catheter lumen) and a radially expanded condition (e.g. to radially engage the lumenal wall). Various types of expandable stents include a tubular or partially tubular wall structure having a network of interconnected struts separated by voids, which structure may be cut from a tube, such as by laser cutting or photoetching, or may be formed by securing adjacent shaped rings. Most common expandable stents are metallic (e.g. the struts). Examples of different types of such expandable stents include: balloon expandable (e.g., stainless steel, or cobalt-chrome); and those which are self expanding (e.g., nickel-titanium alloy such as Nitinol™). Stents may also be non-metallic, such as polymeric. Stents may also be constructed as a helical or otherwise folded ribbon structures reconfigurable between collapsed and expanded conditions for delivery and implantation, and may be formed in a composite structure with other materials such as grafts to form stent-grafts (e.g. for treating abdominal aortic aneurysms).

Stents may be used to maintain lumenal patency, such as for example those currently used in peripheral, coronary, and cerebrovascular vessels, the alimentary, hepatobiliary, and urologic systems, the liver parenchyma (e.g. porto-systemic shunts), and the spine (e.g., fusion cages). Conventional stents are typically greater than about 2 to 3 millimeters, though smaller stents are contemplated, such as in particular for certain particular indications. For example, stents may be used in the interstitium to create conduits between the ventricles of the heart and coronary arteries, or between coronary arteries and coronary veins. In the eye, stents may be used for the Canal of Schlem to treat glaucoma. Stents also may be used in order to occlude a lumen, such as for example to occlude fallopian tubes for fallopian tubal ligation, feeder vessels to tumors, or aneurysms; such occlusive stents typically include the addition of bioactive material such as fibrin to cause an occlusive thrombosis. Occlusive stents may be expanded within the lumen to be occluded, or may be contracted around the lumen from outside the vessel wall.

The term “electroforming” refers to a process in which electrochemical deposition processes are performed on a sacrificial substrate. After the deposition process, the substrate is etched away, leaving a freestanding structure.

### II. Methods of Manufacture

Embodiments of the invention include methods of manufacturing bioactive composite materials. In one embodiment, the method includes providing an electrochemical solution comprising metal ions and a bioactive material. The electrochemical solution may be an electroless deposition bath that is formed using metal salts, a solvent, and a reducing agent, or a electrodeposition bath which is formed with a cathode (the substrate for deposition), an anode, and an electrolyte solution containing the metallic ions to be reduced. Complexing agents, stabilizers, and buffers may also be present in the bath. After the electrochemical solution is formed, a substrate contacts the electrochemical solution. For example, the substrate may be immersed in a bath comprising the electrochemical solution.

Prior to contacting the electrochemical solution, the substrate can be prepared for the electrochemical process. In one preparation step, an anodic process is performed. In this process, the substrate is submerged in a hydrochloric acid bath. Current is passed through the solution, creating small pits in the substrate. Such pits promote adhesion. Also, a sensitizing agent and/or catalyst can be deposited on the substrate to assist in the creation of nucleation centers leading to the formation of the bioactive composite structure. Loosely adhered nucleation centers can also be removed from the surface of the substrate using, for example, a rinsing process.

After contacting the electrochemical solution, a bioactive composite structure is formed on the substrate using an electrochemical process. The electrochemical process may be an electrolytic or an electroless process (i.e. electro- or electroless deposition.) After forming the bioactive composite structure, the bioactive composite structure/substrate combination is removed from the bath containing the electrochemical solution.

After removing the bioactive composite structure/substrate combination from the bath, the combination may be further processed if desired. For example, in some embodiments, a topcoat may be formed on the bioactive composite structure. Additional details about the topcoat and other subsequent processing steps are described below.

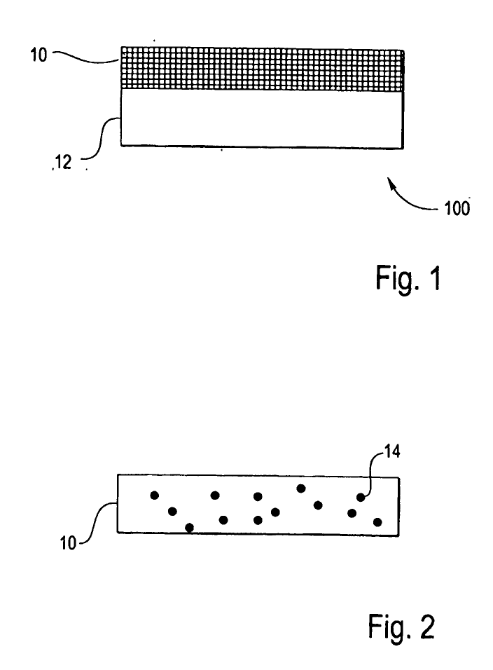

A device including a bioactive composite structure according to an embodiment of the invention is shown in FIGS. 1 and 2. The Figures depict a device 100 including a bioactive composite structure 101 including a metal matrix 10 and the bioactive material 14 within the metal matrix 10. The bioactive composite structure 101 is on a substrate 12. The proportion of bioactive material to the proportion of metal in a bioactive composite structure is high relative to the proportions of bioactive material that might be found in conventional bioactive composite structures, containing a metallic matrix.

Embodiments of the invention have a number of other advantages over conventional methods for forming bioactive composite structures. First, when bioactive materials are incorporated into a metallic matrix using an electrochemical process, the electrochemical process does not damage the bioactive material. Unlike high temperature processes for forming metallic matrices (e.g., sintering), embodiments of the invention can be performed at temperatures that do not harm bioactive materials (e.g., proteins). Second, in some embodiments of the invention, bioactive materials are more easily loaded into a metallic matrix than in conventional metallic matrices. For example, problems associated with impregnating a preformed metallic matrix with a solution comprising a carrier and a bioactive material are generally not present in embodiments of the invention. Consequently, the bioactive composite structures according to embodiments of the invention can have higher proportions of bioactive materials than conventional bioactive composite structures. Third, in some embodiments, the formed bioactive composite structure releases a bioactive material in a very localized area at specified times in an active and/or passive fashion over a period of months to years. The controlled and/or predictable release of the bioactive material can be achieved using embodiments of the invention. Fourth, when the bioactive composite material is in the form of a layer on a metallic substrate, the bioactive composite material and the metallic substrate can have similar properties. For example, the ductility and the modulae of elasticity of the bioactive composite material can be substantially the same as the underlying substrate. In another example, the metallic matrix of the bioactive composite structure and the substrate can both be metallic in embodiments of the invention. They can have similar thermal expansion coefficients, thus decreasing the likelihood that the two materials may separate due to thermal expansion differences. Fifth, the bioactive composite structures can be made uniform in composition and thickness in embodiments of the invention. If the bioactive composite structure is in the form of a layer on a metallic substrate with a complex shape, the layer can easily conform to the complex shape. Other advantages of embodiments of the invention are provided below.

### A. Substrate Preparation

Any suitable substrate may be coated using embodiments of the invention. The substrate may be porous or solid, and may have a planar or non-planar surface (e.g., curved). The substrate could also be flexible or rigid. In some embodiments, the substrate may be a stent body, an implant body, a particle, a pellet, an electrode, etc.

The substrate may comprise any suitable material. For instance, the substrate may comprise a metal, ceramic, polymeric material, or a composite material. Illustratively, the substrate may comprise a metal such as stainless steel or nitinol (Ni-Ti alloy). Alternatively, the substrate may comprise a polymeric material including fluoropolymers such as polytetrafluoroethylene. In some embodiments, the substrate may comprise a sacrificial material. A sacrificial material is one that can be removed, for example, by etching, thereafter leaving a free-standing bioactive composite structure.

The substrate may be prepared in any suitable manner prior to forming a bioactive composite structure on it. For example, the substrate surface may be sensitized and/or catalyzed prior to performing an electroless deposition process (if the surface of the substrate is not itself autocatalytic). Metals such as Sn can be used as sensitizing agents. Many metals (e.g., Ni, Co, Cu, Ag, Au, Pd, Pt) are good auto catalysts. Palladium (Pd), platinum (Pt), and copper (Cu) are examples of “universal” nucleation center forming catalysts. In addition, many non-metals are good catalysts as well.

Before forming the bioactive composite structure, the substrate may also be rinsed and/or precleaned if desired. Any suitable rinsing or pre-cleaning liquid or gas could be used to remove impurities from the surface of the substrate before performing the electrochemical process. Also, in some embodiments involving electroless deposition, distilled water may be used to rinse the substrate after sensitizing and/or catalyzing, but before performing the electrochemical process in order to remove loosely attached molecules of the sensitizer and/or catalyst. In addition to, or in place of this, an anodic, or sometimes cathodic, cleaning process is used in some embodiments to produce pits which enhance adhesion.

### B. Electrochemical Processes

In embodiments of the invention, an electrochemical deposition process is used to form the bioactive composite structure. Electrochemical deposition processes include electrolytic (electro) deposition and electroless deposition.

In embodiments of the invention, a bioactive material is incorporated into an electrochemical bath along with a source for metal ions. The bioactive material can include any of the particular materials mentioned above as well as other materials. For example, the bioactive material refers to any organic, inorganic, or living agent that is biologically active or relevant. The bioactive material could also comprise biologically active molecules such as drugs. In embodiments of the invention, the bioactive material may be soluble or insoluble in the electrochemical solution.

The bioactive material may also comprise particles (e.g., in the size range of 0.1 to about 10 microns). The particles may comprise the bioactive material in a crystallized form. Alternatively, the particles comprise a polymer, ceramic, or metal, which can store a bioactive material. The particles are preferably insoluble in the electrochemical solution. In this case, a particulate stabilizer such as a surfactant could be added to the electrochemical solution to improve the homogeneity of the particles in the solution.

Without being bound by theory, it is believed that when performing an electrochemical deposition process according to some embodiments, nanometer-sized crystallites (crystallized metal atoms) and the bioactive material “co-deposit”. At first, the process occurs on the surface of the substrate. Following the deposition of tens of nanometers of metal, the co-deposition occurs on the already deposited metal. Thus, the bioactive material and the metal atoms may deposit substantially simultaneously. When co-depositing metal atoms and the bioactive material, the bioactive material is incorporated into the metal matrix. These crystallites confine the bioactive material in the formed bioactive composite structure.

By co-depositing the bioactive material along with the metal, the concentration of the bioactive material in the bioactive composite structure is high. Moreover, the problems associated with impregnating porous structures with bioactive materials are not present in embodiments of the invention. In embodiments of the invention, the bioactive material substantially fills the voids in the metal matrix so that the loading of the bioactive material in the metal matrix is maximized.

As noted, electrochemical processes include electrolytic (electro) and electroless deposition processes. In electrolytic (electro) deposition, an anode and cathode are electrically coupled through an electrolyte. As current passes between the electrodes, metal is deposited on the cathode while it is either dissolved from the anode or originates from the electrolyte solution. Electrolytic deposition processes are well known in, for example, the metal plating industry and in the electronics industry.

An exemplary reaction sequence for the reduction of metal in an electrodeposition process is as follows:
M^{Z+}solution+ze→M_{lattice (electrode)}

In this equation, M is a metal atom, M^{z+} is a metal ion with z charge units and e is an electron (carrying a unit charge). The reaction at the cathode is a (reduction) reaction and is the location where electrodeposition occurs. There is also an anode where oxidation takes place. To complete the circuit, an electrolyte solution is provided. The oxidation and reduction reactions occur in separate locations in the solution. In an electrolytic process, the substrate is a conductor as it serves as the cathode in the process. Specific electrolytic deposition conditions such as the current density, metal ion concentration, and bioactive material concentration can be determined by those of ordinary skill in the art.

Electroless deposition processes can also be used to form a bioactive composite structure. In an electroless deposition process, current does not pass through the solution. Rather, the oxidation and reduction processes both occur at the same “electrode” (i.e., on the substrate). It is for this reason that electroless deposition results in the deposition of a metal and an anodic product (e.g., nickel and nickel- phosphorus).

In an electroless deposition process, the fundamental reaction is:
M^{z+}solution+R_{ed solution}>M_{lattice(catalytic surface)}+Oxₛₒₗᵤₜᵢₒₙ

In this equation, R is a reducing agent, which passes electrons to the substrate and the metal ions. Ox is the oxidized byproduct of the reaction. In an electroless process, electron transfer occurs at substrate reaction sites (initially the nucleation sites on the substrate; these then form into sites that are tens of nanometers in size). The reaction is first catalyzed by the substrate and is subsequently auto-catalyzed by the reduced metal as a metal matrix forms.

The electroless deposition solution can comprise metal ions and a bioactive material. Suitable bioactive materials are described above. The solvent that is used in the electroless deposition solution may include water so that the deposition solution is aqueous. Deposition conditions such as the pH, deposition time, bath constituents, and deposition temperature may be chosen by those of ordinary skill in the art.

Any suitable source of metal ions may be used in embodiments of the invention. The metal ions in the electrochemical solution can be derived from soluble metal salts before they are in the electrochemical solution. In solution, the ions forming the metal salts may dissociate from each other. Examples of suitable metal salts for nickel ions include nickel sulfate, nickel chloride, and nickel sulfamate. Examples of suitable metal salts for copper ions include cupric and cuprous salts such as cuprous chloride or sulfate. Examples of suitable metal salts for tin cations may include stannous chloride or stannous floroborate. Other suitable salts useful for depositing other metals are known in the electroless deposition art. Different types of salts can be used if a metal alloy matrix is to be formed.

The electrochemical solution may also include a reducing agent, complexing agents, stablizers, and buffers. The reducing agent reduces the oxidation state of the metal ions in solution so that the metal ions deposit on the surface of the substrate as metal. Exemplary reducing compounds include boron compounds such as amine borane and phosphites such as sodium hypophosphite. Complexing agents are used to hold the metal in solution. Buffers and stabilizers are used to increase bath life and improve the stability of the bath. Buffers are used to control the pH of the electrochemical solution. Stabilizers can be used to keep the solution homogeneous. Exemplary stabilizers include lead, cadmium, copper ions, etc. Reducers, complexing agents, stabilizers and buffers are well known in the electroless deposition art and can be chosen by those of ordinary skill in the art.

Illustratively, a nickel-phosphorous alloy matrix can be electrolessly deposited on a substrate along with a bioactive material such as a drug. The substrate may need to be activated and/or catalyzed (using, e.g., by Sn and/or Pd) prior to metallizing. To produce this alloy matrix, a typical electroless deposition solution contains NiSO₄ (26 g/L), NaH₂PO₂ (26 g/L), Na-acetate (34 g/L) and malic acid (21 g/L). The solution may be in the form of a bath and may contain ions derived from the previously mentioned salts. A bioactive material is also in the bath. In this example, sodium hypophosphite is the reducing agent and nickel ions are reduced by the sodium hypophosphite. The temperature of the bath is from room temperature to 95° C. depending on desired plating time. The pH is generally from about 5 to about 7 (these processing conditions could be used in other embodiments). The substrate to be coated is then immersed in the solution and a bioactive composite structure can be formed on the substrate after a predetermined amount of time. The Ni ions in solution deposit onto the substrate as pure nickel (reduction reaction) along with nickel-phosphorous alloy (oxidation reaction); the bioactive material co-deposits along the crystallite and grain boundaries of the deposited metal matrix to form a bioactive composite structure. The bioactive material may co-deposit along with nickel atoms. Typically, the amount of phosphorous ranges from <1% to >25% (mole %) and can be varied by techniques known to those skilled in the art.

Although co-deposition of the metal atoms and the bioactive material is preferred, co-deposition is not necessary in some embodiments. For example, in other embodiments, a very thin metallic layer on the order of tens of nanometers can be formed on a substrate. A bioactive material is then either adsorbed, covalently bound, or deposited on top of the nanometer thick metallic layer. Additional metallic layers are subsequently added afterward. In between metallic layers, additional layers of bioactive material can be adsorbed, covalently bound, or deposited. This type of process produces a dense bioactive composite material.

The metallic matrix of the bioactive composite structure can include any suitable metal. The metal in the metallic matrix may be the same as or different from the substrate metal (if the substrate is metallic). The metallic matrix may include, for example, noble metals or transition metals. Suitable metals include nickel, copper, cobalt, palladium, platinum, chromium, iron, gold, and silver and alloys thereof. Examples of suitable nickel-based alloys include Ni—Cr, Ni—P, and Ni—B. Any of these or other metallic materials may be deposited using a suitable electrochemical process. Appropriate metal salts can be selected to provide appropriate metal ions in the electrochemical solution for the metal matrix that is to be formed.

The metallic matrix may also have voids in a crystal lattice. Typically, the average void size is less than about 1 micron. For example, in some embodiments, the average size of the voids in the metallic matrix may be less than about 100 angstroms (e.g., less than about 10 nanometers). The bioactive material can be incorporated into the voids of the metallic matrix.

In the formed bioactive composite material, the volume percent of the bioactive material is high. For example, in embodiments of the invention, the bioactive material can make up percentage of the bioactive composite structure. Preferably, the bioactive material can make up greater than about 10%, or greater than about 25% percent by volume of the bioactive material.

The bioactive composite structure may be in any suitable form. For example, the bioactive composite material may in the form of a layer on the substrate. The layer may have any suitable thickness. For example, the layer may have a thickness of less than about 100 microns in some embodiments (e.g., from about 0.5 to about 10 microns). In another example, the layer may have a thickness of greater than about 1 mm. In other embodiments, the bioactive composite structure need not be in the form of a layer. For example, the bioactive composite structure could be in the form of small particles in some embodiments.

Forming a bioactive composite structure using an electroless deposition process is advantageous. First, by using an electroless deposition process, the size of the crystallites and consequent percentage of bioactive material is controllable. Parameters such as the pH, temperature, and the constituents of the deposition bath can be adjusted by the person of ordinary skill in the art to alter the volume percentage of bioactive material in the formed metallic matrix. Second, using an electroless process, substrates having complex geometries can be evenly coated with a bioactive composite structure. As the solutions are aqueous in nature, viscous effects do not dominate in an electroless deposition process (as compared to coating polymeric substances which are viscous). Third, in an electroless deposition process, deposition conditions are mild, occurring at or near room temperature and at or near body physiologic pH. Bioactive materials are not damaged in the process of forming the bioactive composite material. Fourth, the methods according to embodiments of the invention are economical and scaleable, and are more cost-effective than other methods of forming bioactive composite structures.

### C. Subsequent Processing

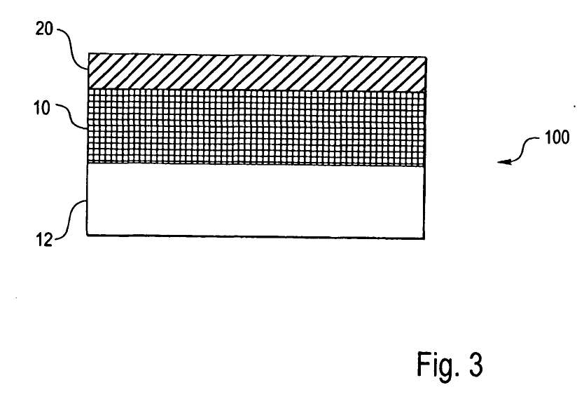

After the bioactive composite structure is formed, it may optionally be further processed in any suitable manner. For example, in some embodiments, a topcoat is formed on top of a bioactive composite structure. FIG. 3 illustrates a device 100 including a bioactive composite structure 10 in the form of a layer in between a substrate 12 and a topcoat 20.

The topcoat can include any suitable material and may be in any suitable form. It can be amorphous or crystalline, and may include a metal, polymer, ceramic, etc. The topcoat may also be porous or solid (continuous).

The topcoat can be deposited using any suitable process. For example, the above-described processes (e.g., electro- and electroless deposition) could be used to form the topcoat or another process may be used to form the topcoat. Alternatively, the topcoat could be formed by processes such as dip coating, spray coating, vapor deposition, etc.

The thickness of the topcoat may vary in embodiments of the invention. For example, in some embodiments, the topcoat may have a thickness greater than about 100 microns. Of course, the thickness of the topcoat can depend on the end use for the device being formed.

In embodiments of the invention, the topcoat may be the only layer on the bioactive composite structure. In other embodiments, any number of suitable topcoat layers may be added to the bioactive composite structure. For example, it is possible that tens to hundreds of individual layers could be formed on the bioactive composite structure (some or all of these layers may be bioactive).

In some embodiments, the topcoat can improve the properties of the bioactive composite structure. For example, the topcoat may include a membrane (e.g., collagen type 4) that is covalently bound to the bioactive composite structure. The topcoat's function can be to induce endothelial attachment to the surface of the bioactive composite structure, while the bioactive material in the bioactive composite structure diffuses from below the topcoat. In another embodiment, a growth factor such as endothelial growth factor (EGF) or vascular endothelial growth factor (VEGF) is present in a topcoat that is on the bioactive composite structure. The growth factor is released from the topcoat to induce endothelial growth while the bioactive composite structure releases an inhibitor of smooth muscle cell growth.

In yet other embodiments, the topcoat can improve the radio-opacity of a medical device which includes the bioactive composite structure, while the underlying bioactive composite structure releases molecules to perform another function. For example, drugs can be released from the bioactive composite structure to prevent smooth muscle cell overgrowth, while a topcoat on the bioactive composite structure improves the radio-opacity of the formed medical device. Illustratively, a topcoat comprising Ni-Cr (nickel chromium) and/or gold can be deposited on top of a bioactive composite structure comprising Ni—P to enhance the radio-opacity of a device incorporating the bioactive composite structure. Underneath the topcoat, a smooth muscle cell inhibitor such as sirolimus is released over a 30-60 day time period from the bioactive composite structure

The topcoat can also be used to alter the release kinetics of the bioactive material in the underlying bioactive composite structure. For example, an electroless nickel-chrome, nickel-phosphorous, or cobalt-chrome coating without bioactive material can serve as a topcoat. This would require the bioactive material to travel through an additional layer of material before entering the surrounding environment, thereby delaying the release of bioactive material. The release kinetics of the formed medical device can be adjusted in this manner.

Alternatively, the topcoat comprises a polymeric material (or other material). In this case, a bioactive material that is the same or different than the bioactive material in the bioactive composite structure may be included in the topcoat. For example, when the topcoat comprises a polymeric storage and release medium, the bioactive material therein can release quickly (e.g., days) from the topcoat, while the material in the bioactive composite structure is released over a period of months to years. In this embodiment, the medical device that is formed may include the combination of a topcoat comprising a polymeric storage and release medium, and a metallic storage and release medium.

Suitable polymers in the topcoat are preferably biocompatible (i.e., they do not elicit any negative tissue reaction) and can be degradable. Such polymers may include lactone-based polyesters or copolyesters, for example, polylactide, polycaprolacton-glycolide, polyorthoesters, polyanhydrides; poly-aminoacids; polysaccharides; polyphosphazenes; and poly (ether-ester) copolymers.

Nonabsorbable biocompatible polymers may also be used in the topcoat. Such polymers may include, for example, polydimethylsiloxane; poly(ethylene-vinylacetate); acrylate based polymers or copolymers, e.g., poly(hydroxyethyl methylmethacrylate); fluorinated polymers such as polytetrafluoroethylene; and cellulose esters.

In yet other embodiments, the topcoat that is on the bioactive composite structure can be a self-assembled monolayer (SAM). The thickness of the self-assembled monolayer may be less than 1 nanometer (i.e., a molecular monolayer) in some embodiments. In one example, a thiol based monolayer can be adsorbed on a nickel matrix of a bioactive composite structure through the thiol functional group and can self-assemble on the nickel matrix. The introduction of the self-assembled monolayer can permit different surface ligands to be used with the bioactive composite structure. That is, various ligands or moieties can be attached to the ends of the molecules in the monolayer that extend away from the bioactive composite structure.

In another embodiment, after forming the bioactive composite structure on a substrate, the substrate can be removed. This could be done to electroform a free-standing bioactive composite structure. For example, as noted above, when forming a medical device, a bioactive composite structure can be formed on a substrate. However, instead of leaving the substrate in the final medical device, the substrate may be etched to remove it from the formed bioactive composite structure. For example, the substrate may comprise an etchable material. Etchable materials include metals such as aluminum or copper or polymeric substances.

The substrate is a sacrificial substrate and can be used as a mandrel for forming a free-standing bioactive composite structure. After etching the substrate, a free-standing bioactive composite structure is formed. Stents, for example, can be formed in this manner. Details regarding the formation of stents using sacrificial substrates are found in U.S. Pat. No. 6,019,784. This U.S. Patent is herein incorporated by reference in its entirety.

The free-standing bioactive composite structure may have dimension on the order of nanometers (e.g., nanoparticles) to meters. For example, the thickness of the free-standing bioactive composite structure may be less than about 1 mm thick. As in other embodiments, a topcoat could be formed on a free-standing bioactive composite structure.

### III. Releasing Bioactive Material From a Bioactive Composite Structure

The bioactive composite structures according to embodiments of the invention can be present in medical devices that are used in vivo. They can be implanted in the body of a patient when used, or could be used external to the body of a patient. In such medical devices, the long term release of a bioactive material from the bioactive composite material is desirable in some instances.

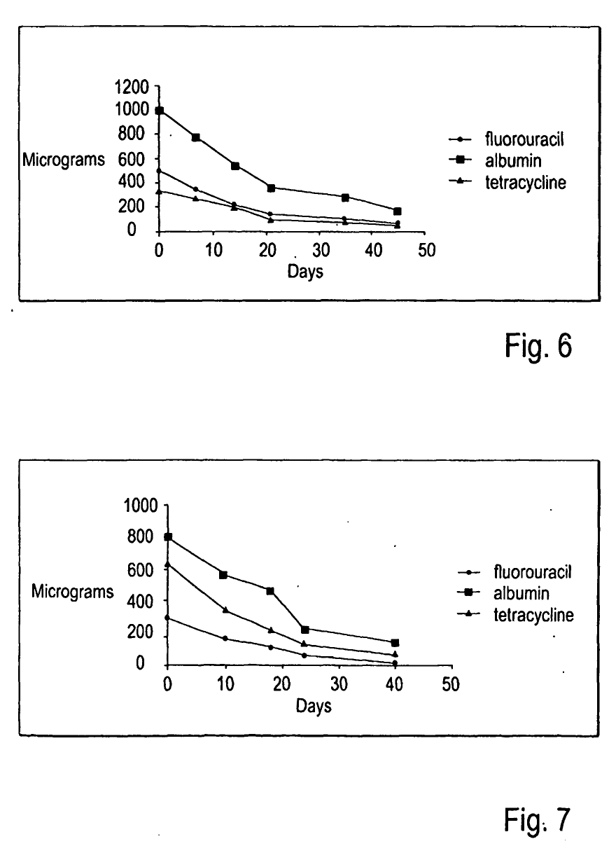

In some embodiments, the bioactive material can diffuse from the metallic matrix in the bioactive composite structure. FIGS. 6 and 7 (described in further detail below) show the results of experiments using embodiments of the invention. As shown in FIGS. 6 and 7, in embodiments of the invention, drugs can be released over long periods of time (e.g., greater than about 10 or about 20 days). Again, without being bound by theory, the release mechanisms in the examples shown in FIGS. 6 and 7 are indicative of simple diffusion. The bioactive material diffuses through the metallic matrix, that is, between individual crystallites and grain boundaries. The bioactive material exchanges places with the components of the metallic film and then diffuses into liquid at the interface of the metallic film and liquid.

Alternatively, the metallic matrix of the bioactive composite structure can erode to release the bioactive material in it. For example, the metallic matrix can be susceptible to electrolytic corrosion. The metallic matrix of the bioactive composite structure can serve as an anode, which results in corrosion of the metallic matrix when current is passed through a circuit which includes the composite structure as an anode. As a result of the corrosion process, the bioactive material is liberated from the metallic matrix. This is useful both in vivo and in vitro. By using a corrosion process, small, controllable quantities of a bioactive material (e.g., a drug or DNA) can be released in a highly localized regions at specified times within a patient or within a diagnostic assay.

Corrosion can occur actively or passively. In an active corrosion process, current is actively applied to the bioactive composite structure using an external power source to corrode the metallic matrix. In a passive corrosion process, the oxidation of the matrix metal of the bioactive composite material can be caused by the difference between the electrical potential of the metallic matrix and an adjacent metal or solution. For example, galvanic corrosion is caused when two metal pieces, in electrical contact with each other, or two adjacent metal areas are at different electrochemical potential. The two metal parts will constitute a galvanic cell, in which the metal part with the lowest electrochemical potential (i.e., the more active metal) will corrode.

In another embodiment, mechanical energy such as ultrasonic energy is applied to the bioactive composite structure. The mechanical energy hastens the rate of diffusion of the bioactive material from the bioactive composite structure. In this embodiment, the metallic matrix may or may not erode. In the case of a stent or other implanted medical device, ultrasonic energy may be applied non-invasively to a patient so that the release of the bioactive material from the stent can occur at a desired time. For example, the application of ultrasonic energy can be, for instance, days, weeks, or months after the stent is implanted.

### IV. Medical Devices

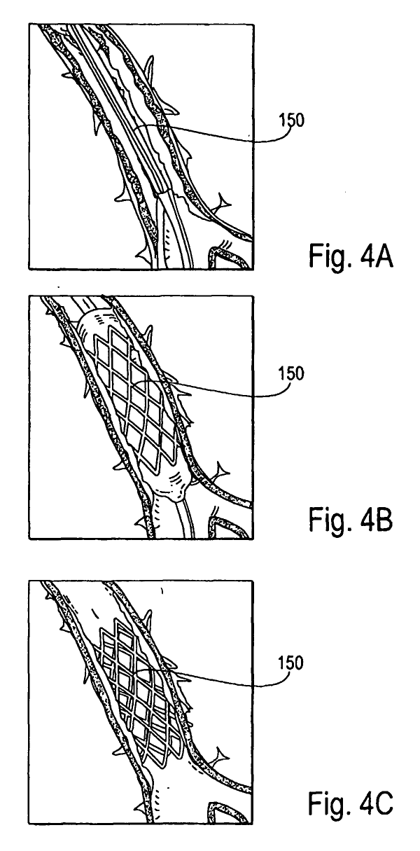

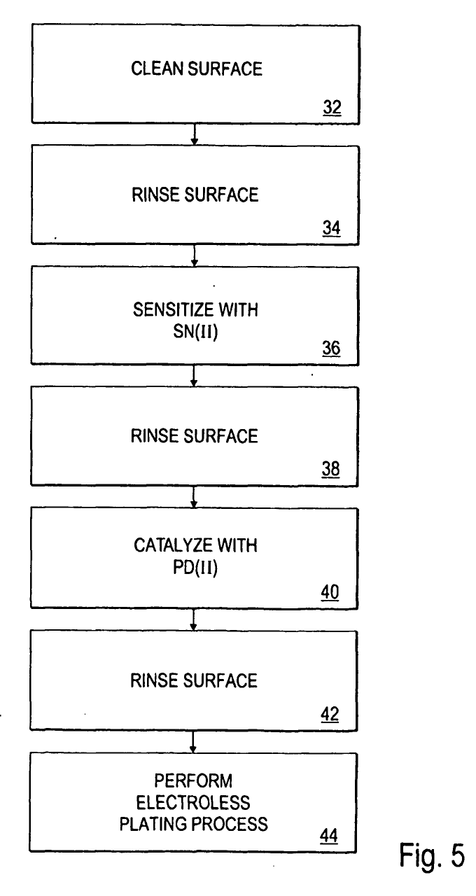

Embodiments of the invention include any suitable medical device incorporating the bioactive composite structure. For example, medical devices according to embodiments of the invention include stents, orthopedic implants, cardiovascular implants, electrodes, sensors, drug delivery capsules, surgical clips, micromechanical systems, and nanomechanical systems. A schematic drawing of a stent 150 in an artery is shown in FIGS. 4(a)-4(c).

In other embodiments, the bioactive composite structures are applied to blood or tissue contacting medical devices, which are dependent on endothelialization of the implant surfaces for biocompatibility. These devices include ventricular assist devices (VADs), total artificial hearts (TAHs), and heart valves. In comparison to stents, which have discontinuous surfaces (e.g., wire meshes with windows), these devices have continuous surfaces. They rely on cell seeding from the bloodstream. Accordingly, the bioactive composite structures can comprise growth factors. The bioactive composite structures provide an attachment surface that could facilitate the attachment and subsequent growth processes of endothelial cells on the surface. Such growth factors include any of a host of integrins, selectins, growth factors, and peptides, which can assist and hasten cell migration and adhesion.

The bioactive composite structures could also be used in drug release devices such as ingestible pills or devices capable of traveling in the bloodstream. These devices can take the form of a sphere, square or cylinder of sufficient size to fit into a body cavity. They can be placed in the human body transcutaneously or orally. Subsequent release occurs from the metallic matrix by one of the methods described above. This type of drug storage and delivery system can be produced in combination with other delivery vehicles such as biodegradeable polymers.

In another embodiment, the bioactive composite material may be present in wells or channels in a microchip-type device. The bioactive composite material in the wells or channels can be covered with a topcoat that is erodable. For example, the metallic matrix of the bioactive composite structure may comprise nickel or a nickel alloy, while the topcoat comprises gold. Electrical current is selectively applied to the gold topcoat, thereby causing it to erode. As a result of the erosion process, the bioactive material is free to diffuse out of each well or channel. Alternatively, the release of bioactive material from each well or channel can be induced by an electrical current. Passive corrosion can be induced by a bimetallic EMF (electromotive force) created by the combination of two metals. Active release can be induced by current induced erosion of the metallic matrix. In both cases, the amount of current applied to the metallic matrix can be directly proportion to the amount of released bioactive material. This design reduces the complexity of such systems compared to current designs.

Aside from use in therapeutic medical devices, the bioactive composite structure can be used in diagnostic devices and bioassays where a precise quantity of bioactive material is required in a spatially and/or temporally controlled fashion. They can be used in the drug discovery process. Bioassays for drug discovery are increasing in complexity and in many cases utilize live cells for bioassays. Modern surface technologies make it possible to study the effects of local chemical gradients in the study of cell response as well as local environmental alterations in cell culture, such as pH. Utilizing embodiments of the invention, dynamic release of bioactive materials at specific places at specific times and in controlled quantities could be used in diagnostic devices and bioassays.

In one embodiment, a bioactive composite structure is formed underneath the surface on which cells are cultured. The bioactive composite structure can be in the form of a pattern with varying concentrations of bioactive materials or in a layer containing one concentration of molecule. When appropriate, the matrix of the bioactive composite structure is dissolved via electrolytic corrosion and the bioactive material is released almost instantaneously into the environment surrounding the cells of interest. The amount of applied current determines the amount of bioactive material released.

This type of technology is meant to mimic the in vivo environment and can be used to study the molecular effects of specific molecules on cells at specific times identified with other biological assays. For example, the affect of molecule X on the cell cycle during G1 or G2, etc. where G1 and G2 are measured with a well-known assay such as a fluorescence assay.

### EXAMPLE I

Six bioactive composite structures were formed. Each bioactive composite structure comprised a nickel-phosphorous metallic matrix formed on a metallic substrate using an electroless deposition process. The substrates used were foils. Three substrates comprised medical grade 316L stainless steel and three substrates comprised nitinol. fluorouracil, tetracycline, and albumin were respectively co-deposited with the nickel-phosphorous on the stainless steel and nitinol substrates.

Each substrate was first prepared using process steps show in FIG. 4. First, the surface of the substrate is cleaned (step 32). Then, the substrate surface is rinsed with distilled water (step 34). After rinsing, the surface of a substrate is sensitized with Sn(II) (step 36). A solution of 0.1 g/L of stannous chloride may be used as a sensitizing solution. After depositing Sn(II) on the surface of the substrate, the substrate is again rinsed with distilled water (step 38) in a second rinse step. Then, a Pd (II) catalyst is deposited on the surface of the substrate. A solution of 0.1 g/L palladium chloride may be used as a catalyzing solution (step 40). The surface of the substrate is again rinsed in a third rinsing step (step 42). Distilled water may be used as the rinsing fluid. After the third rinsing step, the substrate is catalyzed and is ready for electroless deposition. Three stainless steel and three nitinol substrates were prepared using the above described catalyzing process.

Three different electroless plating baths were made. The three different baths were the same, except that the bioactive material was different in each bath. Bath 1 contained 5-fluorouracil, Bath 2 contained tetracycline, and Bath 3 contained albumin. Each bath was at ambient pressure, at a pH of about 7, and at a temperature of about 40° C.

TABLE 1

| Ingredient | Concentration |
|---|---|
| Nickel Sulfamate | 29 g/L |
| Sodium Hypophosphite | 17 g/L |
| Sodium Succinate | 15 g/L |
| Succinic Acid | 1.3 g/L |
| Bioactive material: | 0.25 g/L (Bath 1), 0.25 g/L |
| 5-fluorouracil (Bath 1), | (Bath 2), and 100 ug/ml |
| tetracycline (Bath 2), and | (Bath 3) |
| albumin (Bath 3). | |

Six bioactive composite structures in the form of layers were respectively formed on the substrates (3 stainless steel substrates and 3 nitinol substrates) using electroless deposition (step 44). In general, the time in the bath determines the thickness of the bioactive composite structure. Each substrate was immersed in a bath for about 10 minutes to yield a layer about 4 microns thick. The concentration of the bioactive material in the bath determines the concentration of the bioactive material in the coating. For example, when albumin was used as a bioactive material, the concentration in the coating was 1:10 w/w albumin:metal with 100 ug/ml concentration of albumin in the starting bath.

For each bioactive composite structure, the weight proportion of the bioactive material to the metallic matrix material is listed in Table 2.

The weight proportions of the bioactive materials to the metallic matrices for each bioactive composite material were determined as follows. For each bioactive composite structure/substrate combination, pre- and post-deposition dry weights were measured. After they were formed, each bioactive composite structure/substrate combination was then placed in an electrolytic bath, with the bioactive composite structure being made the anode of an electrolytic circuit. With current introduced into the bath, the metallic matrix of the bioactive composite structure was corroded and passed from the substrate into the electrolytic bath. The amount of the bioactive material in the bath was then optically measured with the use of a spectrophotometer. The numbers below in Table 2 represent the weightₓ/weight _{Ni-p}, wherein the x represents the bioactive material and Ni—P is the electrochemically deposited metal matrix. As shown by the results in Table 2, the concentration of bioactive material to metal is high in each case.

TABLE 2
W/W concentration of bioactive material to Deposited Ni-P Matrix on
Nitinol and 316L Substrates

| | Fluorouracil | Tetracycline | Albumin |
|---|---|---|---|
| Nitinol | 0.100 mg/3 mg | 0.3 mg/4 mg | 0.5 mg/4.8 mg |
| 316L Stainless | 0.4 mg/3 mg | 0.5 mg/4 mg | 0.4 mg/4 mg |
| Steel | | | |

### EXAMPLE 2

Coated stents were formed using the same basic electroless deposition procedure in Example 1. However, in this example, instead of foil substrates, Johnson and Johnson Bx velocity stents (stainless steel) and Johnson and Johnson Smart stents (nitinol) were used as substrates. Bioactive composite structures in the form of layers were formed on the stents.

FIG. 6 shows a graph of the drug elution profiles when Johnson and Johnson Bx Velocity stents (316L stainless steel) were used as substrates. FIG. 7 shows a graph of the drug elution profiles when Johnson and Johnson Smart stents (nitinol) were used as substrates. The amounts on the y-axis of the graphs represent the amount of bioactive material remaining on the stent after elution into a physiologic saline solution.

A similar anodization process as was used in the stent examples as was again applied to the foil substrates. After coating, the coated stent was placed in a physiologic saline solution and the solution changed daily. On the indicated days, the stent coatings were anodized. The amount of bioactive material released in each case was determined using a spectrophotometric assay.

As can be seen in FIGS. 6 and 7, molecules are released from embodiments of the invention over long periods of time. Appreciable amounts of drugs such as fluorouracil, albumin, and tetracycline were released over 40 days. No appreciable corrosion of the coating was observed.

### EXAMPLE 3

Additional experiments were performed in order to further demonstrate the wide range of bioactive materials which can be stored and released in the coating. Table 3 depicts several experiments following the general procedures outlined in example 1, each with one time point and for a new bioactive material. ΔA is the difference in absorbance between an elution bath from the sample containing the respective bioactive material and the absorbance from a sample with pure coating (i.e. control). In this instance, a 1 cm² medical grade 316L stainless steel sample was coated using the above mentioned process. In addition to tetracycline (an antibiotic), Fluorouracil (an antimetabolite), and albumin (a large protein), these experiments depict the ability to store and release rapamycin (a highly lipophilic antirestenosis compound), heparin (a highly hydrophilic, large carbohydrate, anticoagulant molecule), and hydrocortisone (a lipophilic, anti-inflammatory compound).

Table 3 shows the optical absorbance from an elution bath immediately after deposition and after seven days in a 0.9% saline solution. ΔA refers to the absorbance difference between coated with bioactive material and coated without bioactive material. The number in parentheses refers to the characteristic absorbance for each material.

TABLE 3

| | Time = 0, ΔA | Time = 0, 7 days, ΔA |
|---|---|---|
| Rapamycin | 0 | 1.85 (274 nm) |
| Heparin | 0 | 2.4 (230 nm) |
| Hydrocortisone | 0 | 1.2 (250 nm) |

### EXAMPLE 4

The following is a topcoat example. After applying a bioactive coating to a sample of Nitinol (commercially available from Nitinol Devices and Components, Inc), as outlined in example 1, the sample is further processed by placing it in the cathodic position in a second bath containing 100 g/L chromic acid (CrO₃) and 1 g/l H₂SO₄. 200-300 mA/cm² is applied to the sample for about 10 to about 20 seconds to produce a topcoat which delays the diffusion of bioactive material. The chromium topcoat also augments the radiopacity of the device. Under these conditions, release of bioactive material is delayed several days to weeks.

### EXAMPLE 5

Various of the embodiments of the invention, such as according to specific aspects provided above, provide valuable use in coating medical devices notwithstanding the presence or absence of bioactive agents or materials in the coating, and therefore are to be considered broadly beneficial aspects of the invention.

One particular such aspect is illustrated by the following example, wherein nickel release from nickel-titanium alloy is reduced by use of an illustrative coating embodiment of the invention using nickel-phosphorous coating solution and process without bioactive agent.

In a separate experiment, a 1 cm² sample of nickel-titanium alloy was anodized to completely remove the heavy oxide layer on its surface exposing pure nickel titanium. The substrate was subsequently placed into an electroless nickel bath (Bath 1) without a bioactive material. A tremendous autocatalytic reaction was noted on the surface of the nitinol. After 30 seconds, the nickel-titanium substrate was removed from the bath and a shiny coating noted. This coating was not removable by scratching or with scotch tape and showed superior adherence to the nitinol substrate.

The new nickel-phosphorous coated nickel-titanium sample (Ni—P—NiTi) coating was then placed into 1.5 ml 0.9% sodium chloride solution and incubated at 37 degrees for 96 hours after which the sodium chloride solution was removed and replaced with another 1.5 ml and incubated for an additional 96 hours. A parallel control sample of “as-received” nitinol (NiTi) was also incubated at 37 degrees for 96 hours and 192 hours. Atomic Absorption Spectroscopy was used to analyze the nickel content contained in the solution in which the samples were incubated. Results are as follows in parts per million (ppm):

| | Ni-P-NiTi (ppm) | NiTi (ppm) |
|---|---|---|
| Nickel released 96 hours | 15.6 | 19.6 |
| Nickel released 192 | .6 | 1.2 |
| hours | | |

It can be seen that the sample coated with nickel phosphorous resulted in a 25% decrease in the nickel which leached from the nickel-titanium substrate after 96 hours and a 50% decrease in the subsequent 96 hours, both when compared to the uncoated control sample of nickel-titanium substrate.

This benefit derived from coating a nickel-titanium sample according to the invention is exemplary of various broadly beneficial aspects of the invention. In one regard, a substrate comprising nickel is modified to release less nickel than it otherwise would without being treated according to the invention. This is valuable across a wide range of medical devices, in particular implants, which otherwise suffer from nickel release concerns for biocompatibility reasons, in particular regarding patient populations who have nickel allergies. Examples of such medical devices where the present invention provides such value, without requiring incorporation of bioactive agents (or with such agents, if also desired) includes for example all nickel-titanium medical devices, such as according to further illustrative examples stents, filters, wires, or orthodontic devices.

Moreover, the ability to use the same coating and coating process to (a) inhibit release of nickel from such substrates, (b) also provide for coating of bioactive agents, and (c) increase the radio-opacity of the underlying substrate, or any combination thereof, e.g. (a) and (b), (a) and (c), or (b) and (c), is a highly beneficial combination made possible according to the present invention and should be considered an independent, broad aspect of the invention.

Other benefits are apparent according to use of the present invention, with or without inclusion of bioactive agents in a sample formed or coated according to electroless electrochemical deposition process according to the present embodiments. In one particular example, various formulations of coatings and their related processes may be used to enhance the radiopacity of a substrate medical device. More specifically, preparations using relatively radiopaque materials such as chromium for example, e.g. cobalt-chromium combinations, will tend to enhance radiopacity of substrate materials that are relatively less radiopaque, such as for example nickel-titanium alloy substrates, or substrates containing similar radiopaque material(s) but in less dense and therefore less radiopaque proportions. Therefore, coating processes and resulting coated samples having radiopacity enhanced by a coating according to the present embodiments are considered independently beneficial aspects of the invention, with or without inclusion of bioactive agents, and with or without the result of enhanced biocompatibility (e.g. reduced nickel release), though such combinations apparent to one of ordinary skill provide significant further benefit.

Various particular embodiments have been herein described for the purpose of illustrating certain highly beneficial aspects of the present invention. However, many such specific embodiments, despite their specific benefits, should not be considered limiting in all cases and in many regards are exemplary of broader aspects of the invention. For example, specific examples of experiments are herein shown with respect to particular coating processes and results, but other suitable coating formulations, bioactive agents, or the like may be substituted for the specific embodiments described without departing from the intended scope of the invention.

More specifically, nickel-phosphorous coating preparations have been generally used in the experiments recited in the examples to illustrate particular beneficial results. However, other suitable substitute materials may be used in such preparations and still achieve various of the objectives and broad aspects of the invention, such as for example preparations including: one of nickel or phosphorous with suitable substitutes for the other; cobalt; boron; chromium; or other suitable combinations, alloys, or blends of such materials as herein described. Accordingly, the specific combination solutions of nickel-phosphorous is illustrative of broader aspects of the invention encompassing these other substitutes, such as in certain regards to solutions or structures related to: use of metals or other materials forming positive valence ions and reducing agents thereof (e.g. reducing agents of metals); cations and anions; combinations of positive and negative bivalent or trivalent materials; solutions adapted to exhibit electroless electrochemical deposition onto substrates; sterilized structures that are electroless electrochemically formed or coated; etc. These broad aspects illustrated by use of the nickel-phosphorous electrochemical deposition process of the examples include combinations with or without the bioactive agents as either specifically herein described or suitable combinations, blends, or substitutes thereof

In another regard, where particular bioactive agents are specified and used in the experiments of the Examples, these are intended to be illustrative of other compounds of similar characteristics (though the specific agents are related methods and structures are considered of high independent value). For example, tetracycline may in one regard be characterized as an antibiotic agent with respect to certain foreign organisms, and is further characterized as a bioactive agent that is anti-proliferative when it is inhibiting autogenous cell growth, and therefore a possible suitable substitute as an anti-restenosis agent. In another example, 5-fluorouracil is characterized as an mitotic inhibitor as it interferes with DNA replication, mitosis, and cell growth; it is further characterized as being illustrative of the following types of bioactive agents: fluorouracils; uracil analogues; and anti-restenosis agents. Albumin is another compound given specific attention in the present disclosure and via the exemplary experiments, and is characteristic of a large protein, as well as the following types of compounds: peptides; organic molecules; drug carriers; and growth factors. Rap amycin is another bioactive agent herein disclosed in certain particular exemplary embodiments, and is characteristic of compounds that are: highly lipophilic, anti-restenosis agents, and anti-inflammatory agents. Heparin is another such example that is characterized as being: highly hydrophilic; large carbohydrate; anticoagulant agent; carbohydrate growth factor; combined anti-coagulant-antirestenosis agent. Hydrocortisone is yet another example, and is illustrative of compounds having at least the following characteristics: highly lipophilic; and anti-inflammatory agents.

Accordingly, while each one of these bioactive agents represents highly beneficial specific embodiments according to the invention, such other substitutes thereof, e.g. analogs or derivatives of these particular agents, or other substitutes, or combinations or blends between them or incorporating their suitable substitutes, are further considered for inclusion within the broad intended scope of the invention where appropriate according to one of ordinary skill based at least in part upon review of this disclosure.

Still further, it is to be appreciated that the medical device coating and forming methods and results are beneficial in that one coating method and result may be used interchangeably, or in combination, with such varying types of compounds. The various types of compounds that a coating according to certain embodiments of the present invention may be used, interchangeably or in combination, include any one or more (e.g. combinations) of the following types of compounds: organic, inorganic, water soluble, water insoluble, hydrophilic, hydrophobic, lipophilic, large molecules, and small molecules, proteins, mono and polysaccharides, carbohydrates, anti-restenosis compounds, anti-inflammatory compounds, anti-thrombin compounds, anti-metabolite compounds, anti-biotic compounds, etc.

The electroless electrochemical deposition methods herein described, e.g. by reference to the Examples, results in formation of certain metal matrices that possess features that are readily characteristic of such formation process. For example, the metal matrix formed includes a metal in addition to another non-metal material that is derived from a reducing agent as an electron donor to the metal ions formed by the metal in the electrochemical deposition fluid environment. Such combination of materials are not typical chrematistics of metal matrices formed by other deposition methods, e.g. sintering or electroplating. In addition, the structural and size characteristics of the metal matrix formed is characteristic of a process laid down on a molecular, nanometer scale, and results in features such as pore size and other surface characteristics (e.g. smoothness, evenness, etc.) unique to other methods, e.g. versus sintering. Accordingly, it is contemplated that a “metal matrix formed by an electroless electrochemical process,” or other like description, is definitive of a unique and identifiable structure.

In another regard, various additional aspects of the coating methods and observed results related to the embodiments (e.g. by reference to the examples) are further beneficial. For example, the coated stents illustrated by the examples were generally observed to have metal matrix coatings with average thicknesses of less than about 5 microns over the outer surface of the stent struts. Coating of this narrow thicknesses was further observed to hold and elute more than 750 micrograms of bioactive agent in one case, and in another case at least about 1 milligram of bioactive agent. Further observation has revealed that bioactive composite coatings of thicknesses of less than 1 micron, and in many instances as thin as 500 angstroms, may be achieved according to using the methods and materials illustrated by the embodiments.

Structures having such characteristics, e.g. such density of bioactive agent held in and released from a substrate, relative thicknesses, etc., and in particular with respect to bioactive composite structures that provide an anti-restenosis agent on a stent substrate, are independently considered highly beneficial aspects of the invention, without limitation as to the particular coating methods or materials used.

The embodiments herein described by reference to electroless electrochemical deposition are further contemplated for use in combination with other methods, including other coating methods, and in particular other methods for coating metals (e.g. for example sintering or electroplating). For example, a substrate to be coated using electroless electrochemical deposition embodiments of the invention may be initially formed by use of an electroplating, sintering, or other process. Or, such other processes may be used for surface modification of a substrate before, after, or during electroless electrochemical deposition. In this regard, it is contemplated that electroless electrochemical deposition may be used in combination with electroplating deposition, and/or sintering of metals to form structures or coat surfaces.

The various embodiments described herein may be used in combination with radioactive materials, e.g. radioactive metal isotopes, such as for example as coatings on stents or other implants to provide local radiation into tissues. For example, radiation emitting stents may be formed at least in part according to various of the methods and structures herein described in order to radiate lumen walls to prevent restenosis. This may be accomplished instead of, or in combination with, elution of bioactive materials from the stent itself. However, in embodiments where non-radioactive metals are instead used for the metal matrix, benefit is gained by simplicity and other improvement regarding storage and handling, and decreased risks to patient and healthcare provider.

In another similar regard, where reference is herein made to stents in order to illustrate certain embodiments of the invention, other medical devices or other sterile structures or methods are also herein contemplated as suitable substitutes for use.

By further reference to the various illustrative embodiments above, the invention is further considered a broadly beneficial application of electroless electrochemical deposition of materials in order to coat substrates intended to be inserted into a living being, and therefore in a further regard broadly encompasses such processes and coated results in a sterile environment. In one regard, such medical devices according to the invention may be provided non-sterile for later sterilization by an end user or intervening party. However, the various embodiments herein described with respect to medical devices are generally considered to require such sterilization prior to their intended use, and sterile structures incorporating various of the benefits provided by the embodiments above should be considered as independently valuable aspects of the present invention.

The various embodiments have been herein described by reference to highly beneficial electroless electrochemical deposition methods and related structures. However, it is also to be appreciated that many of the problems solved, and beneficial results achieved, according to these highly beneficial embodiments may also be achieved according to other substitute methods, as would be apparent to one of ordinary skill based upon a review of this disclosure. Therefore, despite the independently valuable inclusion of such electroless electrochemical deposition embodiments, such substitutes are to be considered within the broad scope of certain aspects of the invention. For example, structures and methods that provide the coating layers herein described with respect to electrochemical deposition, e.g. including for example a metal composite matrix containing bioactive materials, may be achieved with other substitute methods without departing from the intended scope of such aspects of the invention (e.g. in particular using other processes not requiring sintering, ceramics, or polymers).

For further understanding, electroless deposition process is herein described as a highly beneficial method for depositing a nickel-containing coating onto a nickel-containing substrate, namely for example an illustrative nickel-titanium substrate coated with a nickel-phosphorous coating (that may include bioactive materials). In another example, a coating containing cobalt and chrome, and possibly also containing a bioactive material, may be deposited onto a cobalt-chrome substrate (e.g. a stent), also by use of electroless electrochemical methods as described herein. However, despite the independent benefits provided by such electroless electrochemical methods over other substitutes, such other substitute methods that may provide similar results are considered within the intended scope of the invention with respect to the broad aspects addressing such intended result(s).

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention.

All U.S. Patent Applications, Patents and references mentioned above are herein incorporated by reference in their entirety for all purposes.

1. A method comprising:
(a) providing an electrochemical solution comprising metal ions and a bioactive material;
(b) contacting the electrochemical solution and a substrate; and
(c) forming a bioactive composite structure on the substrate using an electrochemical process, wherein the bioactive composite structure includes a metal matrix and the bioactive material within the metal matrix.

2. The method of claim 1 wherein the metal ions in the electro chemical solution are derived from metal salts, and wherein the electrochemical solution further comprises a reducing agent.

3. The method of claim 1 wherein the electrochemical process is an electroless deposition process.

4. The method of claim 1 wherein the bioactive composite structure is in the form of a layer on the substrate.

5. The method of claim 1 wherein the substrate is a sacrificial substrate, and wherein the method further includes:
(d) removing the sacrificial substrate from the bioactive composite structure.

6. The method of claim 5 wherein the substrate and the bioactive composite structure form a coated stent.

7. The method of claim 1 wherein the bioactive material comprises a drug.

8. The method of claim 1 wherein the matrix comprises nickel, chromium, gold, silver, copper, cobalt, or alloyed combinations thereof.

9. The method of claim 1 wherein the electrochemical process is an electrolytic deposition process.

10. The method of claim 1 further comprising: forming a topcoat on the bioactive composite structure.

11. The method of claim 10 wherein the topcoat comprises a metal.

12. The method of claim 10 wherein the topcoat comprises a polymeric material.

13. The method of claim 10 wherein the topcoat comprises a self-assembled monolayer.

14. A bioactive composite structure comprising:
(a) a metal matrix, wherein the metal matrix is formed using an electrochemical process; and
(b) a bioactive material within the metal matrix.

15. The bioactive composite structure of claim 14 wherein the bioactive composite structure forms a stent.

16. The bioactive composite structure of claim 14 wherein the bioactive composite structure is in the form of a layer on a stent.

17. The bioactive composite structure of claim 14 wherein the bioactive material comprises drugs.

18. The bioactive composite structure of claim 14 wherein the metal matrix comprises a metal alloy.

19. The bioactive composite structure of claim 14 wherein an average void size of the metal matrix is less than about 100 angstroms.

20. The bioactive composite structure of claim 14 wherein the bioactive composite structure is in the form of a layer.

21.-124. (canceled)
